(19) United States
(12) Patent Application Publication
(10) Pub. No.: US 2012/0034131 A1
(43) Pub. Date: Feb. 9, 2012

(54) APPARATUS, SYSTEM AND METHOD FOR PREVENTING BIOLOGICAL CONTAMINATION TO MATERIALS DURING STORAGE USING PULSED ELECTRICAL ENERGY

(76) Inventors: Boris Rubinsky, El Cerrito, CA (US); Michael Belkin, Givat Shmuel (IL); Alexander Goldberg, Jerusalem (IL); Ariel Sverdlik, Tel Aviv (IL)

(21) Appl. No.: 13/259,516

(22) PCT Filed: Mar. 24, 2010
(86) PCT No.: PCT/IL10/00251

§ 371 Date: Sep. 23, 2011

Related U.S. Application Data
(60) Provisional application No. 61/164,502, filed on Mar. 30, 2009.

(51) Int. Cl. A61L 2/03

(52) U.S. Cl. 422/22; 422/243

(57) ABSTRACT

A container having a main storage space for holding a matter, having an inlet/outlet opening and fitted with a PEF system. The PEF system comprises two or more electrodes disposed within said container and contacting the matter; and electric pulse source connected to the two or more electrodes and adapted to pass pulsed electric energy through the matter. The PEF system being activated to deliver a treatment protocol to the matter.

### FIELD OF THE INVENTION

The present invention relates to an apparatus, system and method, using pulsed electrical energy, for preventing materials that come in contact with a living organism from becoming contaminated during storage with biological agents that could be harmful to that organism during storage and use.

### BACKGROUND OF THE INVENTION

### List of References

1. Fuller, G. W., Report on the investigations into the purification of the Ohio River water at Louisville Kentucky. 1898, New York: D. Van Nostrand Company.
2. Sale, A. J. H. and W. A. Hamilton, Effects of high electric fields on microorganisms. 1. Killing of bacteria and yeasts. Biochimica et Biophysica Acta, 1967. 148: p. 781-788.
3. Hamilton, W. A. and A. J. H. Sale, Effects of high electric fields on microorganisms. 2. Mechanism of action of the lethal effect. Biochimica et Biophysica Acta, 1967. 148: p. 789-800.
4. Sale, A. J. H. and W. A. Hamilton, Effects of high electric fields on microorganisms. 3. Lysis of erythrocytes and protopasts. Biochimica et Biophysica Acta, 1968. 163: p. 37-43.
5. Crowley, J. M., Electrical breakdown of bimolecular lipid membranes as an electromechanical instability. Biophys. J., 1973. 13 (7): p. 711-724; Zimmermann, U., G. Pilwat, and F. Riemann, Dielectric Breakdown of Cell Membranes. Biophys. J., 1974. 14 (11): p. 881-899.
6. Miller, L., J. Leor, and B. Rubinsky, Cancer cells ablation with irreversible electroporation. Technology in Cancer Research and Treatment, 2005. 4 (6): p. 699-706.
7. Beebe S J, Fox P, Rec L J, Somers K, Stark R H, Schoenbach K H. Nanosecond pulsed electric field (nsPEF) effects on cells and tissues: Apoptosis induction and tumor growth inhibition. IEEE Transactions on Plasma Science 2002;30:286-92.
8. Doevenspeck, H. Influencing cells and cell walls by electrostatic impulses. Fleishwirtshaft, 1961. 13: p. 986-987.
9. Toepfl, S., et al., Review: Potential of High Hydrostatic Pressure and Pulsed Electric Fields for Energy Efficient and Environmentally Friendly Food Processing. Food Reviews International, 2006. 22: p. 405-423.
10. Lelieved H. L. M., Notermans S., de Haan S. W. H., Food Preservation by pulsed electric fields. From Research to Application. 2007, Cambridge,England: World Publishing Limited
11. Geyer O, Bottone E J, Podos S M, Schumer R A, Asbell P A. Microbial contamination of medications used to treat glaucoma. Br. J. Ophthalmol, 1995; 79 (4):376-379.
12. Schein O D, Hibberd P L, Starck T, Baker A S, Kenyon K R. Microbial contamination of in-use ocular medications. Arch Ophthalmol. 1992; 110 (1):82-85.
13. Stevens J D, Matheson M M. Survey of the contamination of eye drops of hospital inpatients and recommendations for the changing of current practice in eye drop dispensing. Br. J.Ophthalmol., 1992; 76 (1):36-38.
14. Leung E W, Medeiros F A, Weinreb R N, Prevalence of ocular surface disease in glaucoma patients. J Glaucoma., 2008; 17 (5): 350-355.
15. Xiong C et al. A rabbit dry eye model induced by topical medication of a preservative benzalkonium chloride. Invest Ophthalmol Vis Sci, 2008; 49 (5):1850-1856.
16. Dietlein T S, Jordan J F, Lüke C, Schild A, Dinslage S, Krieglstein G K., Self-application of single-use eyedrop containers in an elderly population: comparisons with standard eyedrop bottle and with younger patients. Acta Ophthalmol. 2008; Epub ahead of print.
17. Dart J K, Radford C F, Minassian D, et al. Risk Factors for Microbial Keratitis with Contemporary Contact Lenses A Case-Control Study. Ophthalmology 2008, Jul 1.

Materials which are prone to undesirable organic contamination are sterilized prior to storage. For example one method for sterilization is pasteurization. Pulsed electrical fields have been used for sterilization since the 19^{th} century (1). A very thorough study on the parameters of pulsed electrical fields that destroy microorganisms for sterilization was described in a series of three fundamental papers by Sale and Hamilton in the 1960's (2, 3, 4). Substantial research has been done over the last fifty years to produce better fundamental understanding of the mechanism of cell damage during the application of an electrical pulse on living organisms (5). Moreover, the range of electrical fields and electrical parameters that destroy cells has been expanded in recent years (6, 7).

Patents on devices and methods for using pulsed electrical fields for sterilization and destruction of microorganisms are known for over fifty years. One of the first patents U.S. Pat. No. 3,265,605 on the use of pulsed electrical fields for sterilization of meat was in Germany by Doevenspeck, H. (8). The use of pulsed electrical fields for sterilization of solid and fluid matter has become a well established method in the food industry (9).

Several examples of methods and devices for IRE are known. U.S. Pat. No. 5,462,644 discloses a method of killing microorganisms which form a biofilm on surfaces, including the surfaces of medical articles or on tissue or implant surfaces in a living subject. Killing of biofilm microorganisms is accomplished by applying an electric field to an electrically conductive medium in which the biofilm is contained. The electrically conductive medium either includes a biocide or is capable of generating a biocide in situ upon application of an electric field.

WO9949561 disclosed a high-voltage pulse generator system particularly suited for Pulsed Electric Field (PEF) treatment of food products. The system includes, for example, a power source, an energy storage component in circuit communication with the power source and for storing energy from the power source, a plurality of switches for opening and closing periodically to discharge the energy storage component, and a load comprising at least one PEF treatment chamber in which biological cells are subjected to PEF treatment.

CN101147613 relates to high-voltage pulse electric-field sterilization treatment equipment for liquid food. It is characterized by that the pulse signal output end of its pulse signal generation circuit is connected with control signal input end of pulse generation circuit, two pulse signal output ends of said pulse generation circuit are connected with two pulse control signal input ends of its discharge circuit, the voltage signal input end of said discharge circuit is connected with voltage signal output end of its boost up circuit, two electrode plates of its sterilization condenser are respectively connected with discharge signal output end of discharge circuit and power supply, and its treatment chamber is a sealed rectangular space, two electrode plates of sterilization condenser are respectively fixed on two large-area opposite inner walls of said treatment chamber interior, and on a side wall of said treatment chamber a material inlet is opened. It can be used in the field of liquor food sterilization.

U.S. Pat. No. 5,690,978 discloses a pulsed electric field treatment device for the sterilization and preservation of pumpable food products.

U.S. Pat. No. 5,514,391 discloses methods and apparatuses for preserving fluid foodstuffs. More particularly, it is directed to methods and apparatuses for extending the shelf life of perishable fluid foodstuffs such as dairy products, fruit juices and liquid egg products, which contain significant levels of microorganisms.

U.S. Pat. No. 4,695,472 discloses methods and apparatus for preserving fluid food products by subjecting the fluid foodstuffs such as dairy products, fruit juices and fluid egg products to controlled, pulsed, high voltage electric field treatment.

JP2007229319 discloses a device for performing sterilization in a short time, while suppressing the temperature rise of an object to be sterilized such as a food material.

CN1615759 discloses high voltage pulse electric field processing device for food and beverage consisting of two electrodes connected to two poles of high voltage pulse power supply. The two electrodes are two coaxial hollow pipes in different diameters and the same cross section shapes and are separated with insulating rings on two ends and sealed with insulating discs. The ring interval between two hollow tubular electrodes is cavity for the material to be treated, and there are material inlet and material outlet in the outer layer electrode. The high voltage pulse electric field processing device is suitable for sterilizing food and beverage at normal temperature and artificial ageing of wine, and produces no change of treated material in flavor, state and nutritive components. As wine artificially ageing device, the present invention has fast ageing speed, convenient and efficient treating process.

U.S. Pat. No. 6,787,105 disclose a process and apparatus for reducing microorganisms in a conductive medium using a low voltage pulsed electrical energy. Various drugs leave the manufacturing facility after sterilization. Liquid drugs for example are kept in containers, in which they are stored for periods of time after production. Contamination of these liquid drugs occurs in many circumstances with substantial detrimental effects to the health of the user. There are several possible scenarios in which the products may become not sterile. The first may be related to residual contamination during manufacturing and possible microorganism growth even when the containers are sealed and not in contact with the environment. Another such example of infections engendered by microorganisms are ophthalmic medications contaminated owing to use by a patient, interaction with the environment and incubated for several days/weeks and in particular those used chronically for many weeks, as in glaucoma patients.

Among the methods for avoiding microorganism growth during storage are refrigeration and the addition of chemical additives. Refrigeration is not always effective, in particular during long term storage. Refrigeration can also be detrimental to the composition of the preserved compound, for instance by inducing coagulation of organic molecules. Furthermore, it requires an infrastructure that can support refrigeration. In addition, refrigeration is dependent on the behavioral pattern of the users. The use of chemical additives to drugs that are in potential contact with the environment during storage is also very common. Typically, growth of microorganisms in mediums such as medication, vaccines or cosmetic substances is reduced by the inclusion of preservatives therein, for example, benzalkonium chloride (BAK) used in ophthalmic medication. However, contamination rates as high as 40% are still determined in the presence of preservatives (11,12). Moreover, most preservatives may cause considerable side effects, particularly when applied chronically over long periods of time (13,14,15).

Although application of preservative-free medications seems to produce fewer complications to the user, it is appreciated that such medications have relatively short life span when opened. A possible solution to the use of preservative free, non-refrigerated drugs is to ensemble the drugs into sterilized single use units, which are used immediately after opening. These, however, are considerably more expensive and require a long term supply of such single unit dose medications (16).

Although the aforementioned examples refer to ophthalmic medication, it should be appreciated that the same complications apply to other type of medications, cosmetic substances, crèmes, vaccines etc.

Another situation involving contamination of matter during storage is storage of solid objects in a fluid filled chamber. For example, the storage of contact lenses or prosthesis (e.g. teeth, eye(s) etc.) or medical implants, in a chamber with saline or other fluids. Often the fluids in that chamber become contaminated leading to infections (17). Similarly, medical instruments and devices may become contaminated when stored especially for reuse.

Another situation involving contamination of matter during storage involves various foods in solid or liquid form. For instance such fluid foods as milk, water or wine are kept in closed containers after sterilization. Other types of solids such as meat are also kept in closed containers after sterilization. It is often desired to prevent undesirable microbial contamination from occurring in those foods after they are opened to the environment or from residual contamination. Means to this end include keeping the foods at low temperature in refrigeration to reduce microbial metabolism or treating the foods with such means as pasteurization or adding preservatives, such as sulfides. However each of those methods has drawbacks such as the need for refrigeration facilities or the changes in chemical composition induced by pasteurization or addition of additives.

The invention disclosed here presents a solution to storage of matters with the potential for becoming contaminated during the storage without the use of refrigeration or chemical additives.

### SUMMARY OF THE INVENTION

The present invention is directed towards a device, a system and a method adapted for use during storage and prior to use of a matter stored within the container, to reduce or substantially eliminate undesirable organic contamination of the stored matter when there is the potential for the matter to become contaminated during storage without affecting the intended nature of the matter.

Specific to the devices, systems and methods discussed here are that the materials can be stored and sterilized for periods of time, under conditions giving rise to the possibility of undesirable microorganisms contamination. Possibilities for undesirable microbial contamination during storage can occur through various means such as interaction between the materials and a biologically uncontrolled environment or because of residual microorganism inadvertently left after sterilization.

The present invention is directed towards devices, systems and methods adapted to reduce microorganisms in a matter stored therein using electrical pulses. The pulses may be delivered as several series, with each series spaced a part from the other so as to allow the heat produced and pH changes produced by the electrical pulses to dissipate, and with the pulses so designed to cause only irreversible damage to microorganisms (s) contained within the matter without affecting the matter itself.

The present invention is further directed towards a system comprised of the stored matter and the storage container that has the capability to sterilize the matter during storage.

### Terminology And General Principles of the Invention

The term “reduction” as used herein means that the treatment applied on a matter results in mortality of some or all target organisms. In other words, after treatment in accordance with the present invention, the treated matter contains a substantially decreased number of viable microorganisms as compared to control. The microorganism may include bacteria, fungi, protozoa, algae, spores and the like in any of their forms.

The term “matter” includes mediums such as liquid fluids (e.g. emulsions, liquid medications, vaccines, liquid foods etc.), substances of various viscosities (e.g. creams, gels, medical or cosmetic substances), solid(s) in fluid solution (e.g. eye lenses in a preservation medium), solids such as meat etc., which have a potential of becoming microbiologically contaminated and capable of causing harm to those consuming or coming in contact with such medium.

“Microorganism reduction” is achieved by applying pulsed electrical energy having defined voltage, frequency and pulse waveform characteristics to the target microorganisms contained in the matter. The control settings are designed to treat the entire range of possible undesirable microorganisms, for this purpose different parameters of the treatment may be changed during application of treatment protocol.

By the terms “pulsed electrical field” or “pulsed electrical energy” or “pulsed energy” (PEF), it is meant that the combination of electrical field, frequency and pulse waveform applied to the organic contaminants is such that substantially no free radicals are formed, no ionizing radiation is created, no significant temperature rise is detected, no pH change is detected etc. such that by application of electrical pulses no detrimental effect on the matter is detected, namely the pulsed electrical field will affect only the target contaminant, not the matter.

In accordance with the present invention, it is important not to affect the molecular structures of the components in the stored matter and therefore the treatment is delivered in such a way that the parameters to be monitored and controlled are conductivity of the matter within a defined treatment space, pH of the matter, temperature of the mater, voltage potential between the cathode and anode electrodes of the puller, current generated by the electrodes into the matter, pulsed electrical energy, etc.

The term “stored volume” or “storage volume” in accordance with the invention may be a volume of any shape and size suitable for storage of the matter and which contains at least part of the matter.

The term “treatment space” or “treatment volume” in accordance with the invention may be a volume of any shape and size suitable for holding at least part of the matter and subjecting it to pulsed energy. The treatment space is defined by walls of an enclosure comprising at least a pair of electrodes for generating the pulsed energy within the enclosure. The treatment space may coincide at least in part with the treatment volume or at least with the inlet and/or outlet of the treatment volume.

The term “treatment protocol” refers to a protocol applied on the matter within the treatment space comprising a sequence of electrical pulses with various voltages and amplitudes whose outcome is the desired reduction in contamination of matter. An aspect of this invention is that during the storage of matter the treatment protocol is delivered to the treatment space at least one time and may be delivered as needed for the period of storage to handle recontamination or residual contamination during storage. During the application of the treatment protocol, the different parameters of the PEF system may change during application of the protocol.

The term “active agent” in accordance with the invention refers to a pharmaceutically active agent (such as a drug or a vaccine), an agent used for imaging, a cosmetically active agent, or an hygiene—related active agent such as that used for cleaning teeth, contact lenses or any device to be places on or in the body (including medical devices).

In such a case the “matter” is in fact the carrier of the active agent and is in fact a pharmaceutically acceptable carrier (adapted for any type of administration including: oral, intravenous, intra-muscular, ophthalmic, by inhalation, to the ear, topical, sub-cutanous, transdermal etc.); a cosmetically acceptable carrier (in the form of a liquid, a lotion, a cream, a salve, an ointment), or a carrier for oral hygiene, contact lens hygiene, or medical device hygiene purposes.

Different active agents can “tolerate” different elevation of temperature or change of pH before they are damaged. Basically, the more sensitive the active agent, the pulse intensity has to be lower and or the duration between pulses/series of pulses has to be longer to avoid elevation of temperature of the medium or changed in pH or changed in the configuration of molecules in matter.

### Design Principles of the Invention

a) Use of pulsed electrical fields (PEF) for sterilization;
b) The use is for stored matter;
c) The matter is kept in a storage volume;
d) In one implementation the matter undergoes several use cycles each use episode composed of an opening event where material is extracted, a closing event where the container is closed, and a storage period between one closing event and the next opening event. It each opening event the material is exposed to the environment and has a danger of being contaminated. The PEF are delivered at least once every use cycle;
e) In another implementation the matter in the storage volume undergoes at least one treatment cycle, during which the matter is moved to a treatment space and the PEFs are delivered and the matter is then returned to the storage volume;
f) Matter is treated in a treatment volume where the pulsed electrical field is applied;
g) The treatment volume coincides at least in part with storage volume or at least with the inlet and/or outlet to the storage volume;
h) The electrical fields are delivered through at least two electrodes fitted at a vicinity of the treated volume
i) Because of the coinciding volumes of treatment and storage volumes the electrodes are disposed in the storage volume or a portion thereof or at least at the inlet/outlet of the storage volume;
j) Treatment protocols may be delivered once or more and may differ from one another;
k) Sterilization through PEF is based on producing an electrical field in the entire medium or a portion thereof that has the ability to cause electrical field induced damage to microorganisms in the part of the medium that is targeted. The electrical fields can be produced through reacting electrodes in contact with the solution, through salt bridges, through electrical discharge in a solution across a dielectric barrier or by induction electromagnetic field. The electrical fields which are produced can vary as a function of time and space and depend on the electromagnetic boundary conditions set from the exterior of the system as well as the electrical properties of the solution.

In accordance with the above specified principles, the geometry of the container is assessed and the positioning of the electrodes is determined. Then the electrical field is calculated and the ability of the field to sterilize the desired volume is evaluated. The temperature is calculated and the thermal damage to the medium is estimated. The pH changes are evaluated and the pulses are designed to minimize changes to important molecules in matter. The electrical field that may induce chemical changes to important molecules in matter is assessed and the pulses are further designed to minimize changes to important molecules in matter.

One aspect of the present invention is to provide a device and system adapted to reduce microorganisms in the matter stored therein using PEF treatment protocol affecting the target microorganism(s) without causing detrimental effects to the stored matter.

In accordance with one embodiment of this aspect the device and system of the invention comprise a space adapted for long term storage of matter. The device is provided with an opening through which the matter is introduced/dispensed. The storage volume is fitted with a PEF system comprising:
a. two or more electrodes disposed within said space and contacting the matter directly or through a conductive fluid or gel; and
b. an electric pulse source connected to the two or more electrodes and adapted to pass electric current through a treatment volume which constitutes the entire or part of the storage volume.

In accordance with another embodiment of this aspect the device of the invention comprises a main space adapted for holding and long term storage of matter and a dispensing space adapted to hold and dispense a dose volume. The dispensing space is in flow communication with the main space and is provided with a dispensing outlet; and wherein the main space or/and at least the dispensing space is fitted with a PEF system.

In accordance with yet an embodiment of this aspect, the device of the invention comprises a main space adapted for holding and long term storage of matter and a treatment space. The matter is circulated between the main space and the treatment space and the PEFs are applied to the matter in the treatment space during flow of the matter therethrough.

Another aspect of the present invention is to provide a device having a storage volume containing the matter to be treated, a PEF system for microorganism reduction in the matter within the storage volume which affects the target microorganism(s) without causing detrimental effects to the stored matter.

The present invention further concerns a device as described herein containing a fluid holding at least one active agent.

Another aspect of the invention is to provide a system for applying PEF comprising
a container adapted to receive a matter, comprising a space adapted for holding the matter and having an inlet/outlet opening;
a matter;
two or more electrodes disposed within said container and contacting at least a portion of the matter;
electric pulse source connected to the two or more electrodes and adapted to pass electric current through the matter.

According to an embodiment of this aspect, the container further comprises a dispensing space for dispensing of a volume, said dispensing space being in flow communication with the space and formed with a dispensing outlet.

Yet another aspect of the invention relates to a method for reducing microorganisms and comprises the following steps:
Providing a container adapted to hold a medium comprising active agents, fitted with an PEF system comprising two or more electrodes disposed within said container so as to contact the medium; an electric pulse source connected to the two or more electrodes and adapted to pass pulsed electrical energy through the medium; and a control unit connected to the electric pulse source adapted to allow the user to control the pulsed electrical energy output of the system;
Setting the electrical energy of the system; and
Applying a sterilization/treatment protocol that may consist of application of electrical pulses that can range in length, in pulse amplitude, may vary in shape, applied at least once and whose effect is such that it reduces the number of viable microorganisms in stored matter without inducing any change in the matter medium that may damage the active agents contained therein.
The entire sterilization protocol is delivered to the stored medium or portions thereof once or more to facilitate long term storage while interacting with the environment. The parameters of the PEF system may be determined on a case by case basis. Typically this may be done easily by introducing into a model system a fluid containing an active agent of interest such as a drug or a vaccine together with a known amount of a microorganism that can infect the container. Than a series of pulses varying in intensity, duration and frequency are applied and the parameters of the medium that can cause damage to the active ingredient (e.g. temperature, pH, ionic content etc.) are monitored.

The parameters chosen are such that on the one hand do not cause substantial damage to the matter or the active agent, and on the other hand cause a significant reduction of the target microorganism.

Any one or more of the following features/parameters and designs may be applied to any one of the aspects subject of the present invention:
the sterilization/treatment protocol is applied to the stored volume or portions thereof more than once during the period of storage, thereby facilitating sterilization of at least a dispensed portion and optionally of the entire matter, also facilitating long term storage;
the sterilization/treatment protocol is applied to the stored volume or portions thereof at least one time prior to the use of the matter;
There are several use cycles and the sterilization/treatment protocol is applied to the stored volume or portions thereof at least one time after the use of the matter;
There are several use cycles, each use episode composed of an opening event where material is extracted, a closing event where the container is closed, and a storage period between one closing event and the next opening event and the sterilization/treatment protocol is applied to the stored volume or portions thereof at least one every cycle;
the sterilization/treatment protocol is applied to the stored volume or portions thereof at least one time during the period of storage and at least one time prior to the use of the matter;
the device and system of the present invention may be provided with a control unit for monitoring and controlling at least one or more of the following parameters of the system:
   1) verification of pH values—to ensure the medium has not changed any of its characteristics;
   2) temperature—to ensure the applied energy has remained within the limits and has not raised the temperature of the medium;
   3) pulsed electrical energy—is monitored along with voltage monitoring of the electrical pulse source to ensure consistent energy feed conditions for consistent treatment effects;
   4) chemical structure of molecules.
The device and the system may be provided with a timer for automatically activating the PEF system, either at a predetermined time or intermittently, such as to sterilize the medium within the container.
The PEF system further comprises a power supply adapted to apply the sterilization/treatment protocol more than one time. The power supply comprises three components:
   i. A component adapted to produce and apply a pulsed electric field;
   ii. A control system for controlling and/or monitoring the parameters of the PEF, and optionally adapted to activate the treatment protocol at predetermined intervals.
   iii. Electric power source for powering the system, such as mains power system, batteries, alternative power sources such as mechanical power generators, solar power cells, piezo-electric power source, etc.
the power may be applied to the system through a fixed power source, e.g. batteries, electric cord coupled to the mains; a detachable attachable power source, eg. a pack of batteries; or by induction, where the container is received with a cradling member with a suitable induction arrangement therebetween.
the PEF system may be operated either manually or automatically.
the control system is set to deliver a treatment protocol after each use of the device.
the control system is set to deliver a treatment protocol prior to each use of the device.
the at least two electrodes may have equal surface areas, be parallel to each other and/or equidistant from each other.
the at least two electrodes are placed such as to affect the desired volume treated.
a sterilization/treatment protocol can consist of delivery of electrical pulses that can range in length from nanoseconds to seconds, more preferable from nanoseconds to milliseconds and more preferable from 10 microseconds to one millisecond, in pulse amplitude from 50 V/Cm to 100 kV/cm more preferable from 300 V/cm to 60 kV/cm can vary in shape from square pulses to exponential decay pulse can be delivered in number from one pulse to 500 pulses more preferable from 10 to 100 pulses at intervals of from nanoseconds to seconds, more preferable from 10 microseconds to 1 milisecond and whose effect is such that it reduces the number of viable microorganisms without inducing any change in the medium that may damage the active agents contained therein.
The electrical energy of the system may be set to a value in a range between 5.4 kV/cm and 10 kV/cm;
The pulse wave may have a length of 100 μsec and may be delivered at a frequency of 1 Hz.
The pulse may be a square-wave pulse.
The pulses may be delivered as a sequence of 1, 2,3,4,5 . . . 100 pulses and may be delivered continuously or discretely. In the event that the pulses are delivered in a discrete fashion, the pulse(s) are delivered with a time interval therebetween. The time interval may range from nanoseconds to minutes, more preferable from 10 microseconds to 1 millisecond.
twenty pulses may be given in four sets of five pulses separated by one minute interval between the sets.
at least 10 pulses may be delivered continuously.
The device and/or system of the invention may be used by at home, hospitals/clinics, during manufacturing etc.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1 is a schematic sectioned representation of a dosing container according to an embodiment of the present invention;

FIG. 2 is a schematic sectioned representation of a container according to an embodiment of the present invention; with an activation mechanism provided in the cover, not shown;

FIG. 3 is a schematic sectioned representation of a container according to another embodiment of the present invention;

FIGS. 4a-4c are a schematic sectioned representation of a container according to yet another embodiment of the present invention;

FIGS. 5a-5c are a schematic sectioned representation of a outlet/inlet portion of a container according to embodiments of the present invention;

FIGS. 6a-6d are a schematic sectioned representation of systems according to embodiments of the present invention;

FIG. 7 is a schematic representation of a system according to yet another embodiment of the present invention;

FIGS. 8a and 8b show bar charts presenting results for a microorganism survival as a function of the number and sequence of electroporation pulses for an electrical field of 10 V/cm; and

FIGS. 9a and 9b show bar charts presenting the effect of the field intensity on the microorganism survival and solution temperature, respectively.

### DETAILED DESCRIPTION OF EMBODIMENTS

The present invention discloses a device, a system and a method for reducing microorganisms in matter utilizing PEF for sterilization of matter. Embodiments of the invention may be defined in accordance with several groups, as follows:

I. A container adapted to contain and store matter comprising a PEF system with the electrodes fitted within the body of the container and in which the PEF sterilization/treatment protocol will be applied on the volume of matter stored therein at least once to sterilize and maintain the matter sterile;
II. A container adapted to contain and store matter comprising a PEF system with the electrodes fitted at an inlet/outlet portion of the container storing the matter to prevent the stored matter from becoming contaminated through the inlet outlet;
III. A container adapted to contain and store matter comprising a PEF system with the electrodes fitted at an inlet/outlet portion of the container storing the matter to prevent contaminated stored matter from contaminating an organism using the stored matter.
IV. A container adapted to contain and store matter having a storage space and a treatment space, comprising at least one PEF system where the electrodes are fitted at least at the treatment space such that when matter is propelled through the treatment space, the PEF system is activated and the matter is treated/sterilized.
V. A container containing matter and adapted to store the matter comprising at least one PEF system for sterilizing the stored matter within the container.

In the figures, like reference numerals indicate the same elements throughout.

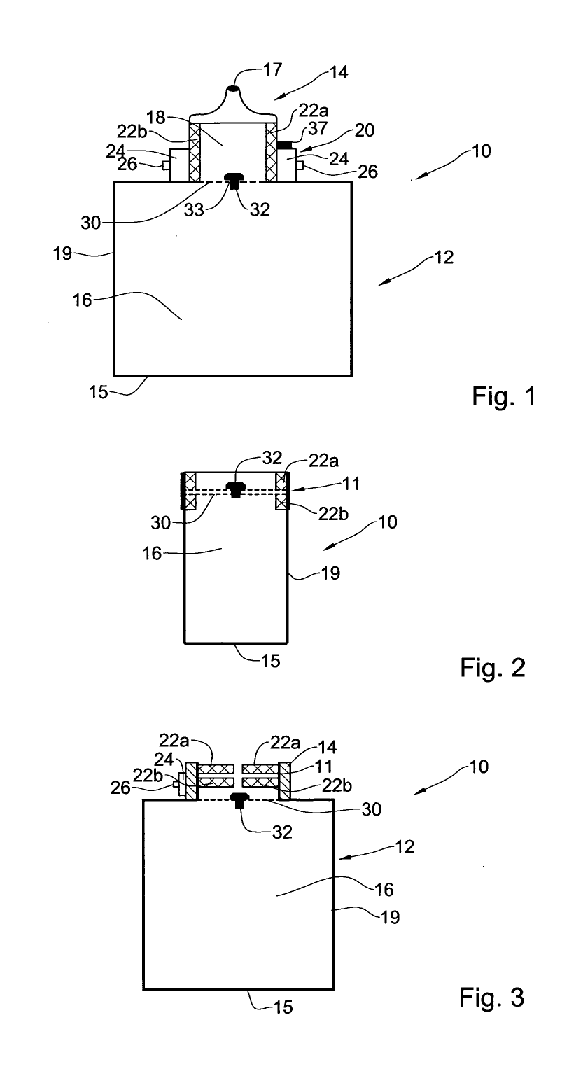

Attention is first directed to FIGS. 1 to 4 of the drawings, illustrating a “real-time” sterilization container, in accordance with one aspect of the invention, generally designated 10. In accordance with one example, the container comprises a main body 12 having a space 16, a secondary body 14 having a dispensing space 18 having a bottom portion 13 connected to the main body 12 such as to be in the fluid flow communication with the space 16, and adapted for holding at least one dose of a medium therein and a top portion provided with a dispensing aperture 17 for dispensing the medium therethrough and a PEF system, generally designated 20, fitted between the secondary body 14 and the main body 12 such as to allow sterilization at least of the medium in the vicinity thereof.

The container as illustrated in FIGS. 1-3 is further provided with a membrane 30 fitted with a one way valve 32, such as a mushroom type valve, allowing the medium to flow through an opening 33 in the membrane towards and into the dispensing space 18 when the container 10 is for example turned over or tilted to a side.

It will be appreciated that the container may also be fitted with any type of dosing mechanism extending through the membrane so as to pump a metered dose of medium from the reservoir of the medium in the main body 12 into the dispensing space 18 of the secondary body 14, thus eliminating the need in turning over or tilting the container.

The PEF system 20 comprises two electrodes 22a and 22b disposed on the inside of the secondary body 14, so as to come in contact with the medium when contained within the dispensing space 18, an electric pulse generating source 24 in contact with the electrodes 22a and 22b. The electric pulse generating source 24 comprises a power source (e.g. battery, a rechargeable battery, conductive chargeable capacitor etc.), pulse controller, adapted to generate pulsed electric energy and an activation mechanism 26 adapted to activate the electric pulse generating source 24 so as to close an electric circle between the electrodes and the electric pulse generating source 24 and discharge pulsed electric energy.

According to an example of the invention, the PEF system is further provided with a control unit 37 connected to the electric pulse source adapted to allow the user to control the values of the pulsed electrical energy output of the system.

According to the example illustrated in FIG. 1, the main body 12 of the container 10 and the secondary body 14 are integrally formed. According to the examples illustrated in FIGS. 2 and 3, the main body of the container comprises a base 15, side wall(s) 19 and an attachment portion 11. According to these examples, the secondary body 14 is an add-on member and may be mounted on the main body 12 of the container 10 through the attachment portion 11 thereof. According to these examples the attachment portion 11 of the main body is fitted with the electrodes and the membrane fitted with the one-way valve, whilst the electric pulse generating source 24 and the activation mechanism are fitted on the secondary body 14 such that when the secondary body 14 is mounted on the main body 12 the electric pulse generating source 24 and the electrodes are in contact so as to allow activation of the electrodes.

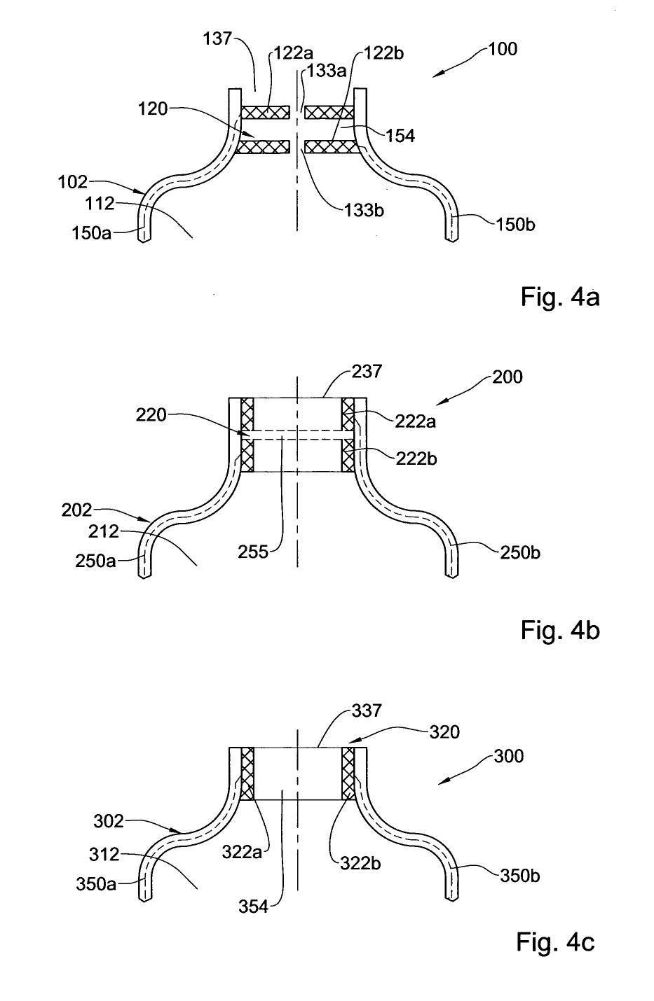

Attention is now directed to FIGS. 4a to 4c of the drawings illustrating a different concept, wherein the PEF system is provided at inlet/outlet of the container.

Referring first to FIG. 4a there is illustrated a neck portion 100 of a liquid holding container 102 (partially illustrated) with a PEF system 120 comprising a first electrode 122a and a second electrode 122b each fitted with a dispensing 133a and 133b respectively, constituting together a flow path between the storage space 112 within the container and the outlet/inlet 137 thereof. Each of the electrodes 122a and 122b is coupled through a conductive segment 150a and 150b to an electric pulse generating source (not shown).

In this example a treatment space 154 extends between two electrodes 122a and 122b whereby a liquid medium dispensed through the outlet 137 flows through said space 154 whereupon it is sterilized/treated prior to dispensing thereof.

The example illustrated in FIG. 4b differs from the example of FIG. 4a in that the neck portion 200 of the container 202 (partially illustrated) is fitted with a different PEF system 220. The PEF system 220 is constituted by a first electrode 222a and a second electrode 222b both of a substantially cylindrical cross section and substantially coaxially disposed within the neck portion 200 each of which is coupled through a conductive segment 250a and 250b to an electric pulse generating source (not shown) (in the examples of FIGS. 4a to 4c the conductive segments are in the form of conductive wires embedded within or along the containers body).

In FIG. 4b, a diaphragm member 255 in a form of a porous filter is provided between the two electrodes 222a and 222b thereby constituting a barrier to prevent a 30 free flow of the medium from the storage space to the inlet/outlet 237 so as to ensure that the dispensed medium undergoes sterilization/treatment protocol.

The example of FIG. 4c discloses a neck portion 300 of a container 302 (partially illustrated) fitted with a modified PEF system 320. The PEF system 320 is constituted by a pair of electrodes 322a and 322b each coupled to an electric pulse generating source (not shown) through a conductive segment 350a and 350b, respectively. The electrodes are disposed substantially opposite one another defining a treatment space 354 with a flow path extending between the storage space 312, through the treatment space 354 and out through the outlet 337.

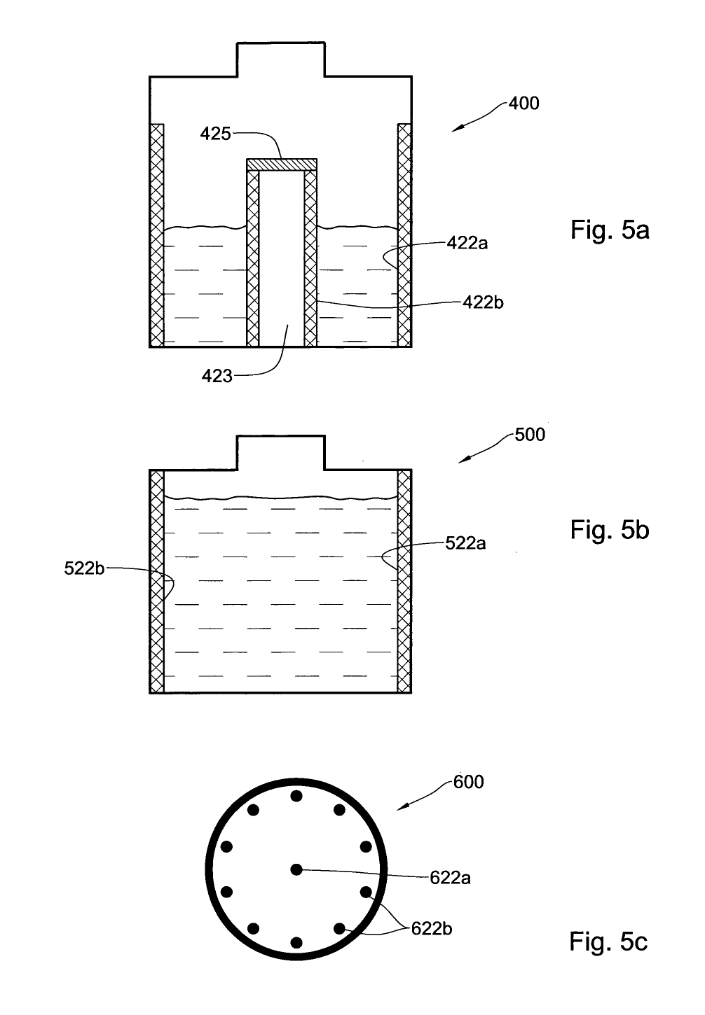

Further attention is directed to the examples illustrated in FIGS. 5a to 5c showing three examples of matter holding containers fitted with a PEF system received within the container. In the example of FIG. 5a there is illustrated a container 400 with a PEF system in the form of a coaxial pair of electrodes 422a and 422b each coupled to an electric pulse generating source (not shown). In this example the coaxial electrodes are cylindrical with the space 423 extending within the electrode 422b being sealed by seal 425 to prevent matter from entering said space without being treated. The example illustrated in FIG. 5b describes a container 500 fitted at its interior with a PEF system comprising a pair of substantially opposed electrodes 522a and 522b for treating a liquid matter stored within the container.

FIG. 5c is a schematic top sectioned view of the container 600 fitted at its interior with a PEF system comprising a central first electrode 622a and surrounded by a plurality of substantially parallely extending second electrodes 622b for treating a liquid matter stored within the container.

The container in accordance with any of the examples provided herein is suitably made of a non conductive material, such as plastic, ceramics, glass etc., or a combination of materials. The container may be of any geometrical shape and volume.

The electrodes according to the present invention may be molded or otherwise integrated within the container or a treatment space.

According to the present invention, the container described herein with reference to FIGS. 1-3 may be pre-filled prior to being purchased by the user (e.g. FIG. 1) or may be filled by the user with medium acquired separately (e.g. FIG. 2). Prior to the activation of the PEF system on the container, the parameter values of the system are set/adjusted as desired through the control unit connected to the electric pulse source such as to allow the user to control the pulsed electrical energy output of the system. According to an example of the invention, the parameter values of the system are pre-set (e.g. during the manufacturing process). A desired amount of the medium is caused to flow into the dispensing space either by the dosing mechanism or simply by tilting or turning over the container. Electrical pulses are then delivered either continuously or discretely by activating the system and the medium is substantially sterilized.

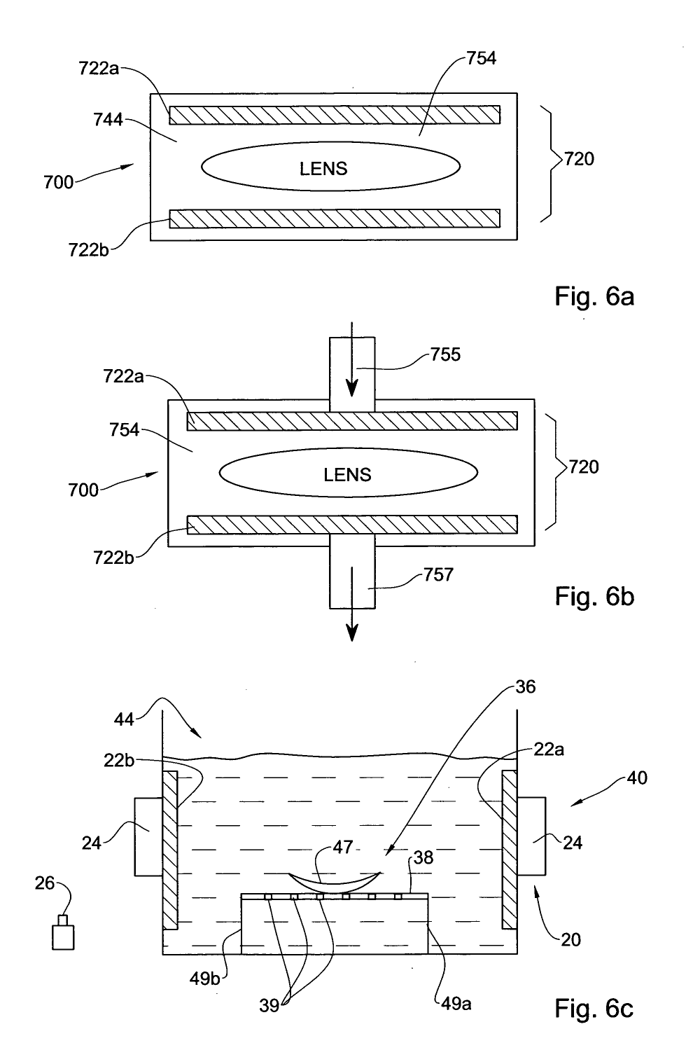

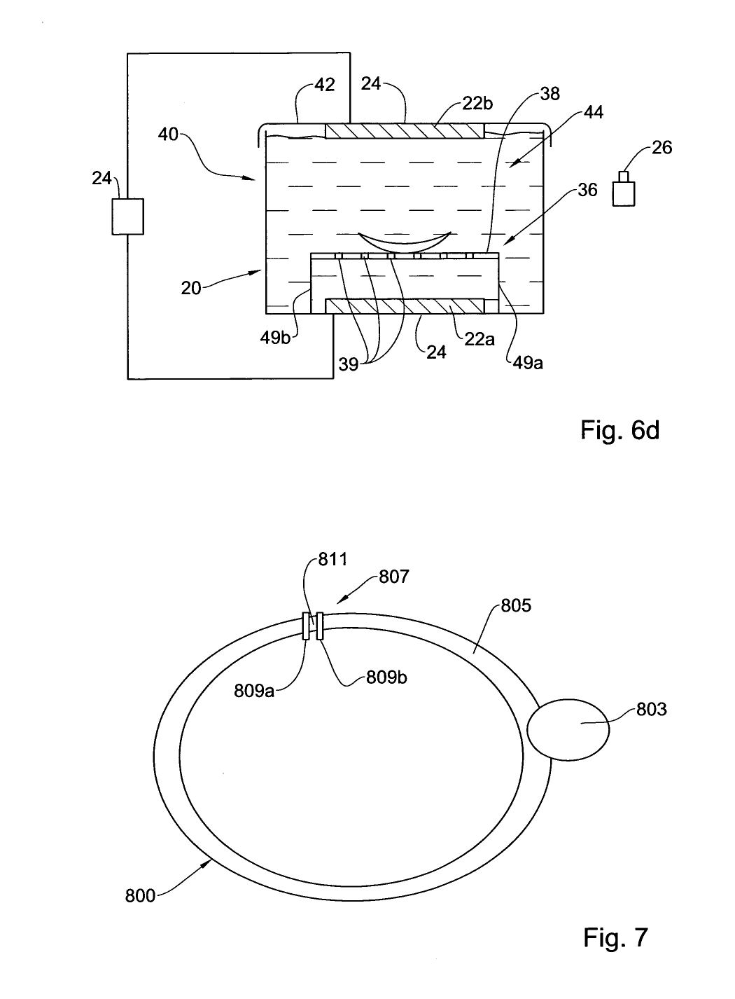

FIGS. 6a-6d illustrate a container, generally designated 700 comprising a main space 744 adapted to receive a medium. FIG. 6a is a schematic illustration of a contact lens storage container fitted with a PEF system 720 comprising pair of electrodes 722a and 722b parallely extending within the container defining between them a treatment space 754. Whilst not seen, the container comprises an electric pulse generating source and a power source.

According to the examples described herein, an activation mechanism and the control unit may be wirelessly connected to the rest of the PEF system so as to allow the user to activate the system from a distance.

The example of the FIG. 6b differs from the example of FIG. 6a by a provision of fluid inlet 755 and a fluid outlet 757 with a flow path extending between the inlet and outlet and a treatment space 754. This arrangement facilitates circulation of fluid through the chamber.

A container 40 illustrated in FIGS. 6c and 6d is further provided with a tray 36 positioned within the main space 44 and immersed within the medium. The tray 36 has a holding portion 38 provided with at least one opening 39 so as to allow fluid to pass therethrough and elevating portion 49a and 49b supporting the tray at an elevated position. FIG. 6d is a modification of the container 40 exemplified in FIG. 6c, having a top cover 42 with one electrode mounted on the base of the container 40 and the other on the inside of the cover 42.

Such container may be used to sterilize semi solid or solid materials immersed in the fluid medium such as contact lens 47.

FIG. 7 illustrates a torus shaped vessel 800 fitted with an intergraded circulation pump 803 for circulating fluid through the interior space 805. Disposed within the torus 800 is a PEF system 807 comprising a pair of electrodes 809a and 809b defining a space therebetween constituting a treatment space 811 and connected to an electric pulse generating source so as to apply a sterilization protocol at least to the medium circulated through the treatment space 811. The electrodes may be in the form of perforated disks to facilitate medium flow circulation through the torus 800. It is appreciated the system exemplified in FIG. 7 may comprises one or more PEF systems disposed along the vessel and further, the pump 803 may be of any type such as a peristaltic pump etc. whereby medium continuously flows through the torus and continuously undergoes sterilization.

The following Example is representative of techniques employed by the inventors in carrying out aspects of the present invention. It should be appreciated that while these techniques are exemplary embodiments for the practice of the invention, those skilled in the art, in light of the present disclosure, will recognize that numerous modifications can be made without departing from the spirit and intended scope of the invention, mutatis mutandis.

### EXAMPLE:

### 1. Pharmaceutical

Preservative free drop solution (HYLO-COMOD® Sodium Hyaluronate 0.1% from URSAPHARM, Arzneimittel GmbH & Co. KG Indutraiestrasse, D-66129, Saarbruken, Germany) was used as a model for a preservative free liquid solution.

### 2. Microorganisms

Escherichia coli tetracycline stable bacteria cultures were used. The cultures of microorganisms were prepared by transferring the organisms from Luria-Bertani (LB) agar plates to 500 ml LB-Miller broth, which was agitated in a temperature controlled incubator at 37° C. until 10⁸ CFU/ml were achieved.

### 3. Electroporation

5 μl of the culture was added to 1 ml of the preservative free drop solution. Several dilutions were performed until the final concentration of microorganism in drops was 10⁶ CFU/ml. Electroporation was performed using a system of the invention. The cells, 80 μl in volume at a concentration of one million cells per milliliter, were placed in the container and subjected to an uni-polar rectangular electrical pulse 100 μsec width with 1 second interval between the pulses.

Immediately after the electroporation the temperature in the cuvette was measured by Reflex Signal Conditioner with 0.7 mm probe covered with polyimide (Neoptix, Inc, Québec, Canada).

The pH of the solution was measured immediately after the electroporation with

Neutralit® pH 5,0-10,0 (MERCK, KGaA, Germany), pH-Indikatorpapier Spezialindikator pH 8.2-10.0 (MERCK, KGaA, Germany), Acilit® pH-Indikatorpapier pH 0.5-5 (MERCK, KGaA, Germany).

### 4. Microorganisms Viability Test

Pour plating counting method was used. After the treatment the solution was dissolved 10 fold in Dulbecco's phosphate buffered saline (Biological Industries, Kibutz Beit Haemek, Israel) in order to eliminate the effect of the eye drops contents on cell growth as described in European Pharmacopoeia Test for efficiency of antimicrobial preservations. 100 μl of each solution was plated in duplicate on LB-Miller Tetracycline agar. Plates were incubated at 37° C. for 18 h. Counting was done using MRC colony counter model 570 (MRC, Israel).

### 5. Experimental Protocol

A study was performed to determine the combinations of PEF parameters in which the fluid drug can be sterilized while atored without a substantial increase in temperature and change in pH. An important aspect of this study was to determine if the drug can be stored in a sterile form using PEF pulses. Furthermore, in the food industry the electrical fields used can be very high, the goal of this study was to determine if lower fields than those used in the food industry are possible so as to allow the system to be used outside laboratory or industry settings.

Electroporation parameters for bacterial sterilization were investigated by comparing the effects of electrical fields of 5.4 kV/cm, 7.2 kV/cm or 10 kV/cm, delivered as 100 μsec length square pulses at a frequency of 1 Hz in sequences of: a) as twenty pulses given four sets of five pulses separated by one minute interval between sets, b) twenty pulses and c) ten pulses. Pour plate counting method was used to determine the bacterial survival percentage after the treatment. The impact of the treatment parameter on temperature and pH was monitored. The end point of each experiment was to measure viability, temperature and pH. All experiments were repeated 5 times.

### 6. Results

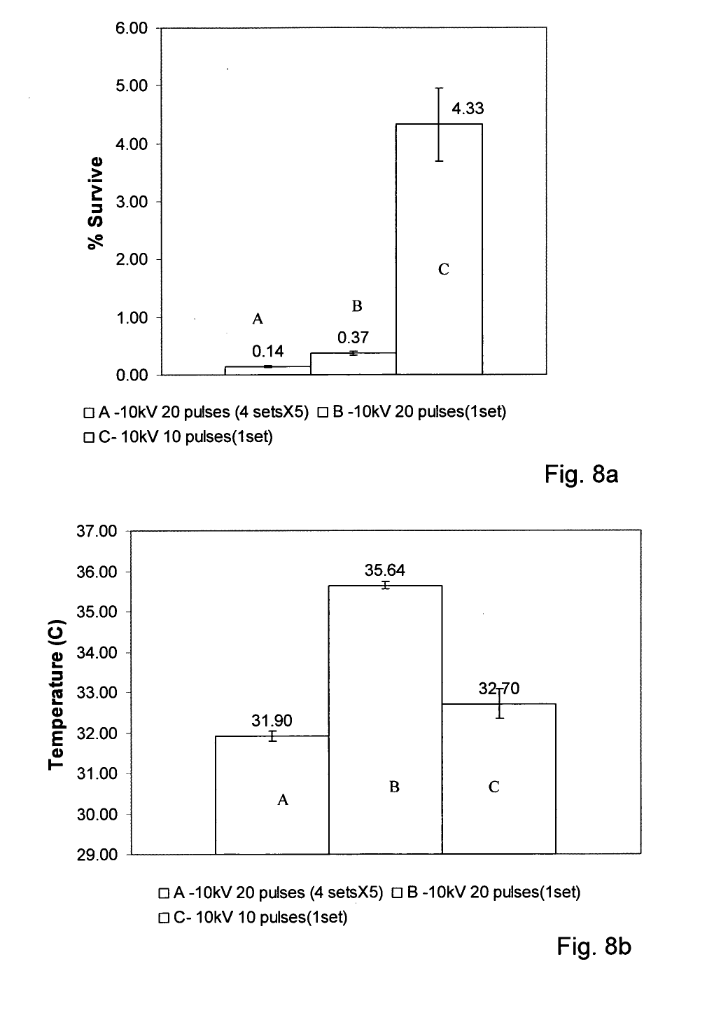

FIGS. 8a and 8b show the effect of the number of PEF pulses and the sequence in which they are delivered, on microorganism survival and solution temperature, respectively. The results are for an electrical field of 10 kV/cm and the pulse sequences of case a (20 pulses—4 discrete sets of 5 pulses), b (20 pulses), and c (10 pulses).

The figures show that doubling the number of pulses from 10 to 20 causes a more than tenfold reduction in the percentage of microorganisms' survival, from 4.33% to 0.37%. Doubling the number of pulses causes only a 3° C. increase in the sample temperature. Changing the sequence in which the 20 pulses are delivered, from continuous in case (b), to discrete four groups of five in case (a), resulted in additional 3 times further decrease of survival number of microorganisms from 0.37% to 0.14%. However, the temperature increased less than when 10 pulses were delivered continuously.

In all the experiments the pH of the samples after treatment remained the same as in the control sample, 7.5.

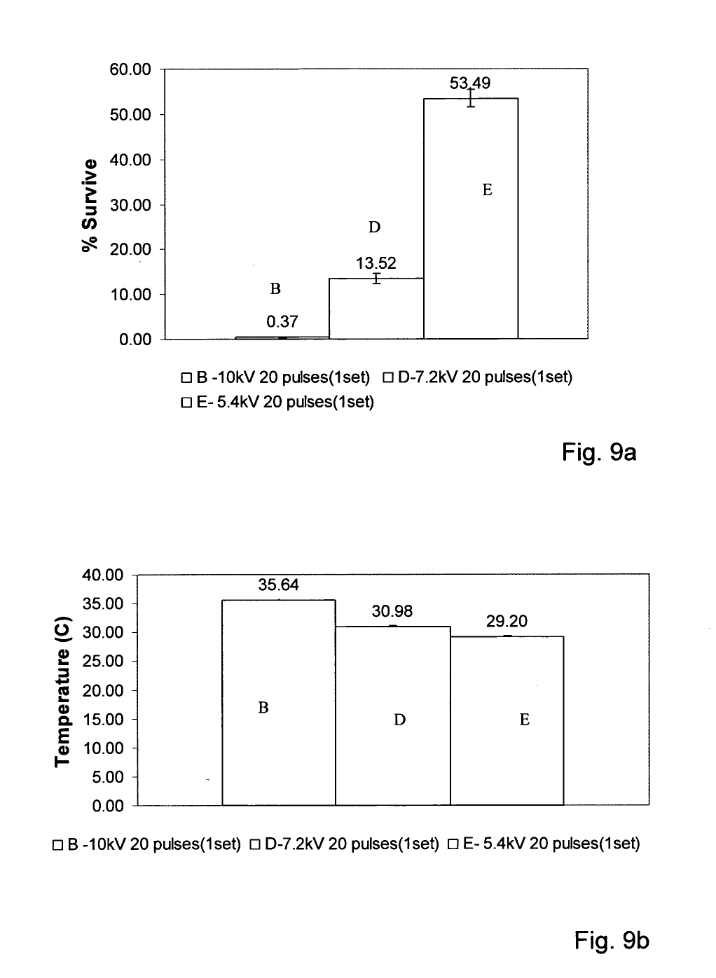

FIGS. 9a and 9b show the effect of the field intensity on the microorganism survival and solution temperature, respectively. The experiments reported here are for a sequence of 20 pulses of 5.4 kV/cm, 7.2 kV/cm and 10 kV/cm, delivered at a frequency of 1 Hz. Increasing the field strength from 5.4 kV/cm to 10 kV/cm caused more than 100 fold reduction in microorganisms' viability from 53.49% to 0.37%. The temperature difference between the highest and lowest field was about 6° C. The highest temperature did not exceed 36 C°. In all the experiments the pH of the samples after treatment was the same as that of the control sample, 7.5.

Those skilled in the art to which this invention pertains will readily appreciate that numerous changes, variations, and modifications can be made without departing from the scope of the invention, mutatis mutandis.

1. A container having a main storage space for holding a matter, having an inlet/outlet opening and fitted with an PEF system comprising:
(a) two or more electrodes disposed within said container and contacting the matter; and
(b) electric pulse source connected to the two or more electrodes and adapted to pass pulsed electric energy through the matter;
wherein, the PEF system is activated to deliver a treatment protocol to the matter.

2. A container in accordance with claim 1, wherein the sterilization/treatment protocol comprises application of electrical pulses at least once such as to reduce the number of viable microorganisms in stored matter without inducing any change in the matter that may damage the active agents contained therein.

3. A container in accordance with claim 1, wherein the treatment protocol is delivered at least once during a storage period of the matter.

4. A container in accordance with claim 1, wherein the treatment protocol is delivered prior to or during its passing through said inlet/outlet opening.

5. A container according to claim 1, wherein the treatment protocol is delivered after its passing through said inlet/outlet opening.

6. A container comprising a main space holding the matter, and a dispensing space of a dosing volume, said dispensing space being in flow communication with said main storing space and formed with a dispensing outlet;
wherein at least said dispensing space is fitted with an PEF system comprising:
(c) two or more electrodes disposed within said container and contacting the matter; and
(d) electric pulse source connected to the two or more electrodes and adapted to pass to pass pulsed electric energy through the matter;
wherein, the PEF system is adapted for sterilization of the matter prior to or during its passing through said dispensing outlet.

7. A container comprising a main space adapted for holding and long term storage of a matter, a treatment space and a PEF system, wherein the matter is circulated between the main space and the treatment space and the PEF system is activated so as to apply PEF to the matter in the treatment space during the circulation of the matter therethrough.

8. A container in accordance with any one of the previous claims, wherein the matter is a liquid.

9. A container in accordance with any one of the previous claims, wherein the matter is a liquid medication.

10. A container in accordance with any one of the previous claims, wherein the matter is a solid.

11. A container in accordance with any one of the previous claims, wherein the matter is a gel.

12. A container in accordance with any one of the previous claims, wherein the matter is a combination of liquid and solid.

13. A container in accordance with any one of the previous claims, wherein the matter is a suspension.

14. A container in accordance with any one of the previous claims, wherein the electric pulse source comprises a power source, pulse controller, an activation mechanism adapted to activate the electric pulse source so as to close an electric circle between the electrodes and the electric pulse source and discharge pulsed electric energy.

15. A container in accordance with claim 14, wherein the power source may be integrated with the container or detachable therefrom.

16. A container in accordance with any one of the previous claims, wherein the PEF system is further provided with a controlling unit for setting and/or adjusting the parameters of the system.

17. A PEF system comprising:
a container adapted to receive an electrically conductive matter, comprising a main space adapted for holding a matter;
electrically conductive matter;
a tray adapted to be positioned within the main space and being at least partially immersed within said matter;
two or more electrodes disposed within said container and contacting the matter;
electric pulse source connected to the two or more electrodes and adapted to pass pulsed electric energy through the matter.

18. A system of claim 17, wherein the matter comprises at least one substantially solid substance and one substantially liquid substance and wherein the at least one substantially solid substance is placed on the tray.

19. A system of claim 18, wherein the at least one substantially solid matter is an contact eye lens and the substantially liquid substance is a contact eye lens preservative medium.

20. A method for reducing microorganisms comprising:
a. Providing a container adapted to hold stored matter fitted with an electroporation system comprising two or more electrodes disposed within said container so as to contact the matter; an electric pulse source connected to the two or more electrodes and adapted to pass pulsed electrical energy through the matter; and a control unit connected to the electric pulse source adapted to allow the user to control the pulsed electrical energy output of the system;
b. Setting the electrical energy of the system;
c. Applying a sterilization/treatment protocol comprising application of electrical pulses at least once such as to reduce the number of viable microorganisms in stored matter without inducing any change in the matter; and
d. The sterilization protocol is delivered to the stored matter or portions thereof once or more to facilitate long term storage while interacting with the environment.

21. A method according to claim 20 wherein the container undergoes a plurality of use cycles each composed of an opening event wherein matter is extracted, a closing event and a storage period between one closing event and the next opening event, wherein the electrical pulse is delivered at least once every use cycle.

22. A method according to claim 20 wherein the electrical pulse is delivered at a period selected from the group consisting of:
a. before or during an opening event
b. after or during a closing event
c. during a storage period
d. at two or more of the periods of (a)-(c).

23. A method according to claim 20, wherein the pulses are delivered continuously.

24. A method according to claim 20, wherein the pulses are delivered discretely.

25. A method according to claim 20, wherein twenty pulses are delivered in four sets of five pulses.

26. A device comprising a storage volume adapted for long term storage of matter having an opening through which the matter is introduced/dispensed, the storage volume being fitted with a PEF system comprising:
a. two or more electrodes disposed within said space and contacting the matter directly or through a conductive fluid or gel; and
b. an electric pulse source connected to the two or more electrodes and adapted to pass electric current through a treatment volume which constitutes the entire or part of the storage volume.

27. A device of comprising a main space adapted for holding and long term storage of matter and a dispensing space adapted to hold and dispense a dose volume, the dispensing space being in flow communication with the main space and provided with a dispensing outlet; and wherein the main space or/and at least the dispensing space is fitted with a PEF system.

28. A device of comprising a main space adapted for holding and long term storage of matter and a treatment space fitted with a PEF system comprising:
two or more electrodes disposed within said space and contacting the matter directly or through a conductive fluid or gel; and
an electric pulse source connected to the two or more electrodes and adapted to pass electric current through a treatment volume which constitutes the entire or part of the storage volume;
wherein, the matter is circulated between the main space and the treatment space and the PEFs are applied to the matter in the treatment space during flow of the matter therethrough.

29. A device having a storage volume containing the matter to be treated, a PEF system comprising:
two or more electrodes disposed within said space and contacting the matter directly or through a conductive fluid or gel; and
an electric pulse source connected to the two or more electrodes and adapted to pass electric current through a treatment volume which constitutes the entire or part of the storage volume;
wherein the PEF system is adapted for microorganism reduction in the matter within the storage volume which affects the target microorganism(s) without causing detrimental effects to the stored matter.

30. A device in accordance with any of the previous claims further containing a fluid holding at least one active agent.

31. A system for applying PEF comprising:
a container adapted to receive a matter, comprising a space adapted for holding the matter and having an inlet/outlet opening;
a matter;
two or more electrodes disposed within said container and contacting at least a portion of the matter;
electric pulse source connected to the two or more electrodes and adapted to pass electric current through the matter.
